(19) United States
(12) Patent Application Publication
(10) Pub. No.: US 2010/0264359 A1
(43) Pub. Date: Oct. 21, 2010

(54) BIOCIDAL MIXTURES

(75) Inventors: Udo Straetmans, Hamburg (DE); Felix Straetmans, Hamburg (DE)

Correspondence Address: LANXESS CORPORATION, 111 RIDC PARK WEST DRIVE, PITTSBURGH, PA, 15275-1112, US

(73) Assignee: LANXESS DEUTSCHLAND GMBH, Leverkusen (DE)

(21) Appl. No.: 12/679,319

(22) PCT Filed: Sep. 24, 2008
(86) PCT No.: PCT/EP2008/062771

§ 371 Date: Jul. 9, 2010

(30) Foreign Application Priority Data
Sep. 26, 2007 (EP) 07117221.7

(51) Int. Cl. C09K 5/04; A01N 43/80; A01N 31/08; A01N 43/42; C07D 275/04; C07C 39/14

(52) U.S. Cl. 252/68; 514/361; 514/732; 514/278; 546/15; 548/209; 568/735

(57) ABSTRACT

The present invention relates to biocidal mixtures containing at least one biocide that is not a polyamine and at least one polyamine, and also to the use of the abovementioned biocidal mixtures in material protection. A special aspect of the invention relates in addition to a method for decolorizing and/or preventing discolorations of pyrithione-containing industrial materials by polyamines.

The present invention relates to biocidal mixtures containing at least one biocide that is not a polyamine and at least one polyamine, and also to the use of the abovementioned biocidal mixtures in material protection. A special aspect of the invention relates in addition to a method for decolorizing and/or preventing discolorations of pyrithione-containing industrial materials by polyamines.

Biocidal mixtures containing at least one biocide and at least one polyamine are known in principle.

For instance, DE 40 33 272 C1 describes a composition containing 1,2-benziso-thiazolin-3-one (BIT) and an amine such as, for example, lauryldipropylenetriamine, which composition in addition contains, as solvent, water and completely water-miscible solvent such as, e.g., butyl diglycol.

GB 1 191 253 discloses aqueous solutions of crude BIT which are known for improving storage stability and have two or more amine compounds from the group diethanolamine, triethanolamine, diisopropanolamine, triisopropanolamine and morpholine.

In addition, GB 1 330 531 discloses mixtures which contain BIT and amines having 2 to 6 carbon atoms, wherein the amines are free from hydroxyl groups and ether groups. Such mixtures are said to have an increased stability against flocculation.

DE 36 099 39 discloses storage-stable liquid preparations of BIT and ethoxylated tertiary amines which contain additionally water, water-miscible solvents such as polyglycols and also other auxiliaries such as complexing agents and/or water softening agents.

Finally, EP 612 470 A discloses disinfectants which contain special glycol ethers and at least one secondary or tertiary amine free from hydroxyl groups. The aforementioned disinfectants are said to have a shortened time of action or improved activity.

Specific biocides are pyrithiones. Pyrithiones are known antimicrobial substances which are used usefully in a multiplicity of applications for preservation. Sodium pyrithione (CAS 3811-73-2) is a water-soluble pyrithione salt having very good antimicrobial properties and is used, e.g., as a preservative (biocide) in industrial process liquids and aids such as, e.g., metalworking fluids, lubricants, binders, adhesives, sealing compositions, leather auxiliaries, paper coating agents and even cosmetic preparations. The production of sodium pyrithione was described for the first time in U.S. Pat. No. 3,159,640.

Unfortunately, sodium pyrithione, as do other salts, e.g. zinc pyrithione, has the property of entering into intensely colored (dark green, blue, black) compounds with heavy metals, especially iron ions. Even if these are only present in traces, e.g. as catalyst residues in chemicals or natural minor components (iron) of additives for paints and varnishes etc., (abovementioned). These discolorations are unacceptable not only for esthetic reasons, but also lead to huge industrial problems such as, e.g., loss of activity.

For example, oil concentrates are subject to a defined specification in the case of color, e.g. brown. A black product is not marketable.

Metalworking fluids are usually used as white emulsions or slightly yellowish solutions. Dark blue to black liquids which color the workpieces and machines black lead to spectacular financial losses. In the past there have been attempts to solve the discoloration which all lead to greater or lesser side effects. In these cases, an attempt is made to displace the heavy metals, in particular iron ions, by other metal ions, or to remove them by complexing. (The reaction of FeIII ions with pyrithione leads to a black precipitate and is thus removed from the system. A loss of biocidal activity is associated therewith). In metalworking fluids, although the use of complexing agents leads to decolorization of Fe-pyrithione, it also reduces the water hardness and virtually always leads to unwanted foam formation of the fluid and foam can neither lubricate nor cool. A further possibility is the use of salts which, with pyrithione, lead to non-colored complex salts, e.g. zinc salts (U.S. Pat. No. 4,161,526). Although this procedure also leads to decolorization, it also leads to a reduction of the biocidal activity of pyrithione and unwanted consequential damage.

Against the background of the abovementioned prior art it was desirable to provide other biocidal mixtures which firstly are distinguished by a reduced fraction of biocides with at least comparable activity and which, in addition, in particular when pyrithiones are used, can retain this activity even in the presence of metal ions.

Biocidal mixtures have now been found containing
at least one biocide that is not an aliphatic primary polyamine and
at least one aliphatic primary polyamine which has at least 5 carbon atoms and does not bear secondary, tertiary or quaternary amino groups,
   with those biocidal mixtures being excepted which contain diaminohexane and 1,2-benzisothiazolin-3-one, as are described in GB 1 330 531.

The expression “aliphatic primary polyamine” is taken to mean in the context of the invention those compounds which comprise at least two primary amino groups which are bound to one sp³-hybridized carbon atom. Typically, such polyamines do not themselves have usable biocidal properties.

It may be mentioned at this point that the context of the invention comprises all optional and possible combinations of the abovementioned and hereinafter listed components, ranges of values, or process parameters mentioned in general or in preferred ranges.

Biocides which are not aliphatic primary polyamines which are used according to the invention are, for example, fungicides, bactericides or algicides or mixtures of such active ingredients, preferably fungicides or bactericides or mixtures of such active ingredients.

Suitable fungicides and/or bactericides are, for example:

aldehydes such as cinnamaldehyde, formaldehyde, glutardialdehyde, β-bromocinnamaldehyde, o-phthaldialdehyde;
benzimidazoles such as carbendazim, benomyl, fuberidazole, thiabendazole or salts thereof;
benzthiazoles such as: 2-mercaptobenzothiazole;
benzthiophene dioxides such as: benzo[b]thiophene-2,2-dioxide carboxylic acid cyclohexylamide;
benzamides such as: 2,6-dichloro-N-(4-trifluoromethylbenzyl)benzamide, tecloftalam;
quinolines such as: 8-hydroxyquinoline and Cu salts thereof;
dithiocarbamates such as cufraneb, ferban, potassium N-hydroxymethyl-N′-methyldithiocarbamate, Na- or K-dimethyldithiocarbamate, mancozeb, maneb, metam, metiram, thiram, zineb, ziram;
formaldehyde and formaldehyde-eliminating compounds such as benzyl alcohol mono(poly)hemiformal, 1,3-bis(hydroxymethyl)-5,5-dimethylimidazolidine-2,4-dione (DMDMH), bisoxazolidines, n-butanol hemiformal, c is 1-(3-chloroallyl)-3,5,7-triaza-1-azoniaadamantane chloride, 1-[1,3-bis(hydroxymethyl-2,5-dioxoimidazolidin-4-yl]-1,3-bis(hydroxymethyl)urea, dazomet, dimethylolurea, 4,4-dimethyloxazolidines, ethylene glycol hemiformal, 7-ethylbicyclooxazolidines, hexahydro-S-triazines, hexamethylenetetramine, N-hydroxymethyl-N′-methylthiourea, methylenebismorpholine, sodium N-(hydroxymethyl)glycinate, N-methylolchloroacetamide, oxazolidines, paraformaldehyde, taurolin, tetrahydro-1,3-oxazine, N-(2-hydroxypropyl)aminomethanol, tetramethylolacetylenediurea (TMAD);
imidazoles such as clotrimazole, bifonazole, climbazole, econazole, fenapamil, imazalil, isoconazole, ketoconazole, lombazole, miconazole, pefurazoate, prochloraz, triflumizole, 1-imidazolyl-1-(4′-chlorophenoxy)-3,3-dimethylbutan-2-one and also metal salts and acid adducts thereof;
iodine derivatives such as diiodomethyl-p-tolylsulfone, 3-iodo-2-propynyl alcohol, 4-chlorophenyl-3-iodopropargyl formal, 3-bromo-2,3-diiodo-2-propenylethylcarbamate, 2,3,3-triiodoallyl alcohol, 3-bromo-2,3-diiodo-2-propenyl alcohol, 3-iodo-2-propynyl n-hexylcarbamate, 3-iodo-2-propynyl cyclohexylcarbamate, 3-iodo-2-propynyl phenylcarbamate;
isothiazolinones such as N-methylisothiazolin-3-one, 5-chloro-N-methylisothiazolin-3-one, 4,5-trimethyleneisothiazolinone, benzisothiazolinone;
methoxyacrylates or the like such as azoxystrobin, dimoxystrobin, fluoxastrobin, kresoxim-methyl, metominostrobin, orysastrobin, picoxystrobin, pyraclostrobin, trifloxystrobin, 2,4-dihydro-5-methoxy-2-methyl-4-[2-[[[[1-[3-(trifluoromethyl)-phenyl]ethylidene]amino[oxy]methyl]phenyl]-3H-1,2,4-triazol-3-one (CAS No. 185336-79-2);
microbicides having an activated halogen group such as bronidox, 2-bromo-2-nitro-1,3-propanediol, 2-bromo-4′-hydroxyacetophenone, 1-bromo-3-chloro-4,4,5,5-tetramethyl-2-imidazoldinone, β-bromo-β-nitrostyrene, chloroacetamide, chloramine T, 1,3-dibromo-4,4,5,5-tetramethyl-2-imidazoldinone, dichloramine T, 3,4-dichloro-(3H)-1,2-dithiol-3-one, 2,2-dibromo-3-nitrilepropionamide, 1,2-dibromo-2,4-dicyanobutane, halane, halazone, mucochloric acid, phenyl(2-chlorocyanovinyl)sulfone, phenyl(1,2-dichloro-2-cyanovinyl)sulfone, trichloroisocyanuric acid;
morpholine derivatives such as aldimorph, dimethomorph, dodemorph, falimorph, fenpropidin, fenpropimorph, tridemorph, trimorphamid and arylsulfonate salts thereof such as, e.g., p-toluenesulfonic acid and p-dodecylphenylsulfonic acid;
naphthalene derivatives such as terbinafine, naftifine, butenafine, 3-chloro-7-(2-aza-2,7,7-trimethyloct-3-en-5-yne);
nitriles such as 2,4,5,6-tetrachloroisophthalodinitrile, disodium cyanodithioimidocarbamate;
phenols such as tribromophenol, tetrachlorophenol, 3-methyl-4-chlorophenol, 3,5-dimethyl-4-chlorophenol, dichlorophen, 2-benzyl-4-chlorophenol, triclosan, diclosan, hexachlorophene, methyl p-hydroxybenzoate, ethyl p-hydroxybenzoate, propyl p-hydroxybenzoate, butyl p-hydroxybenzoate, octyl p-hydroxybenzoate, o-phenylphenol, m-phenylphenol, p-phenylphenol, 4-(2-tert-butyl-4-methylphenoxy)phenol, 4-(2-isopropyl-4-methylphenoxy)phenol, 4-(2,4-dimethylphenoxy)phenol and alkali metal and alkaline earth metal salts thereof;
pyridines and pyrimidines such as ancymidol, buthiobate, fenarimol, mepanipyrin, nuarimol, pyroxyfur, triamirol; 1-hydroxy-2-pyridinethione (and Cu, Na, Fe, Mn, Zn salts thereof), tetrachloro-4-methylsulfonylpyridine, pyrimethanol, mepanipyrim, dipyrithione, 1-hydroxy-4-methyl-6-(2,4,4-trimethylpentyl)-2(1H)-pyridine and pyrithione and salts thereof, such as, in particular, zinc salts thereof and;
quaternary ammonium compounds and guanidines such as benzalkonium chloride, benzyldimethyltetradecylammonium chloride, benzyldimethyldodecylammonium chloride, dichlorobenzyldimethylalkylammonium chloride, didecyldimethylammonium chloride, dioctyldimethylammonium chloride, N-hexadecyltrimethylammonium chloride, 1-hexadecylpyridinium chloride, iminoctadine tris(albesilate);
thiocyanates such as: thiocyanatomethylthiobenzothiazole, methylenebisthiocyanate;
triazoles such as azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, myclobutanil, metconazole, paclobutrazole, penconazole, propioconazole, prothioconazole, simeoconazole, (±)-cis-1-(4-chlorophenyl)-2-(1H-1,2,4-triazol-1-yl)cycloheptanol, 2-(1-tert-butyl)-1-(2-chlorophenyl)-3-(1,2,4-triazol-1-yl)propan-2-ol, tebuconazole, tetraconazole, triadimefon, triadimenol, triapenthenol, triflumizole, triticonazole, uniconazole and also metal salts and acid adducts thereof;
succinate dehydrogenase inhibitors such as benodanil, carboxim, carboxim sulfoxide, cyclafluramid, fenfuram, flutanil, furcarbanil, funnecyclox, mebenil, mepronil, methfuroxam, metsulfovax, nicobifen, pyrocarbolid, oxycarboxin, Shirlan, Seedvax;
sulfenamides such as diclofluanid, tolylfluanid, folpet, fluorfolpet; captan, captofol;
other biocides such as bethoxazin, 5-hydroxy-2(5H)-furanone; 4,5-benzodithiazolinone, 4,5-trimethylenedithiazolinone, N-(2-p-chlorobenzoylethyl)hexaminium chloride, 2-oxo-2-(4-hydroxyphenyl)acetohydroxycinnamoyl chloride, tris-N-(cyclohexyldiazeniumdioxy)aluminium, N-(cyclohexyldiazeniumdioxy)tributyltin or K salts thereof, bis-N-(cyclohexyldiazeniumdioxy)copper, iprovalicarb, fenhexamid, spiroxamine, carpropamid, diflumetorin, quinoxyfen, famoxadone, polyoxorim, acibenzolar-S-methyl, furametpyr, thifluzamide, methalaxyl-M, benthiavalicarb, metrafenon, cyflufenamid, tiadinil, tea tree oil, phenoxyethanol,
preferred fungicides or bactericides are:
azaconazole, bromuconazole, cyproconazole, dichlobutrazole, diniconazole, hexaconazole, metaconazole, penconazole, propiconazole, tebuconazole, dichlofluanid, tolylfluanid, triadimefon, fluorfolpet, methfuroxam, N-methylisothiazolin-3-one, 5-chloro-N-methylisothiazolin-3-one, dichloro-N-octylisothiazolinone, mercaptobenzothiazole, thiocyanatomethylthiobenzothiazole, thiabendazole, benzisothiazolinone, pyrithione and salts thereof, 2-bromo-2-nitro-1,3-propanediol and o-phenylphenol.

Very particular preference is given to N-methylisothiazolin-3-one, 5-chloro-N-methylisothiazolin-3-one, dichloro-N-octylisothiazolinone, mercaptobenzothiazole, thiocyanatomethylthiobenzothiazole, thiabendazole, benzisothiazolinone, pyrithione and/or alkali metal salts thereof and o-phenylphenol, wherein N-methylisothiazolin-3-one, 5-chloro-N-methylisothiazolin-3-one, dichloro-N-octylisothiazolinone, thiabendazole, benzisothiazolinone and o-phenylphenol are still more preferred.

Suitable algicides are, for example:

acetochlor, acifluorfen, aclonifen, acrolein, alachlor, alloxydim, ametryn, amidosulfuron, amitrole, ammonium sulfamate, anilofos, asulam, atrazine, azafenidin, aziptrotryn, azimsulfuron,
benazolin, benfluralin, benfuresat, bensulfuron, bensulfid, bentazone, benzofencap, benzthiazuron, bifenox, bispyribac, bispyribac-sodium, borax, bromacil, bromobutide, bromofenoxim, bromoxynil, butachlor, butamifos, butralin, butylate, bialaphos, benzoyl-prop, bromobutide, butroxydim,
carbetamide, carfentrazone-ethyl, carfenstrol, chlomethoxyfen, chloramben, chlorbromuron, chlorflurenol, chloridazon, chlorimuron, chlornitrofen, chloroacetic acid, chloransulam-methyl, cinidon-ethyl, chlorotoluron, chloroxuron, chlorpropham, chlorsulfuron, chlorthal, chlorthiamid, cinmethylin, cinofulsuron, clefoxydim, clethodim, clomazone, chlomeprop, clopyralid, cyanamide, cyanazine, cycloate, cycloxydim, chloroxynil, clodinafop-propargyl, cumyluron, clometoxyfen, cyhalofop, cyhalofop-butyl, clopyrasuluron, cyclosulfamuron,
diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipropetryn, diquat, dithiopyr, diduron, DNOC, DSMA, 2,4-D, daimuron, dalapon, dazomet, 2,4-DB, desmedipham, desmetryn, dicamba, dichlobenil, dimethamid, dithiopyr, dimethametryn,
eglinazine, endothal, EPTC, esprocarb, ethalfluralin, ethidimuron, ethofumesat, ethobenzanid, ethoxyfen, ethametsulfuron, ethoxysulfuron,
fenoxaprop, fenoxaprop-P, fenuron, flamprop, flamprop-M, flazasulfuron, fluazifop, fluazifop-P, fuenachlor, fluchloralin, flufenacet, flumeturon, fluoroglycofen, fluoronitrofen, flupropanate, flurenol, fluridone, fluorochloridone, fluoroxypyr, fomesafen, fosamine, fosametine, flamprop-isopropyl, flamprop-isopropyl-L, flufenpyr, flumiclorac-pentyl, flumipropyn, flumioxzim, flurtamone, flumioxzim, flupyrsulfuron-methyl, fluthiacet-methyl,
glyphosate, glufosinate-ammonium
haloxyfop, hexazinone,
imazamethabenz, isoproturon, isoxaben, isoxapyrifop, imazapyr, imazaquin, imazethapyr, ioxynil, isopropalin, imazosulfuron, imazomox, isoxaflutole, imazapic,
ketospiradox,
lactofen, lenacil, linuron,
MCPA, MCPA-hydrazide, MCPA-thioethyl, MCPB, mecoprop, mecoprop-P, mefenacet, mefluidid, mesosulfuron, metam, metamifop, metamitron, metazachlor, methabenzthiazuron, methazole, methoroptryne, methyldymron, methyl isothiocyanate, metobromuron, metoxuron, metribuzin, metsulfuron, molinate, monalide, monolinuron, MSMA, metolachlor, metosulam, metobenzuron,
naproanilide, napropamide, naptalam, neburon, nicosulfuron, norflurazon, sodium chlorate,
oxadiazon, oxyfluorfen, oxysulfuron, orbencarb, oryzalin, oxadiargyl, propyzamide, prosulfocarb, pyrazolate, pyrazolsulfuron, pyrazoxyfen, pyribenzoxim, pyributicarb, pyridate, paraquat, pebulate, pendimethalin, pentachlorophenol, pentoxazone, pentanochlor, petroleum oils, phenmedipham, picloram, piperophos, pretilachlor, primisulfuron, prodiamine, profoxydim, prometryn, propachlor, propanil, propaquizafob, propazine, propham, propisochlor, pyriminobac-methyl, pelargonic acid, pyrithiobac, pyraflufen-ethyl,
quinmerac, quinocloamine, quizalofop, quizalofop-P, quinchlorac,
rimsulfuron
sethoxydim, sifuron, simazine, simetryn, sulfosulfuron, sulfometuron, sulfentrazone, sulcotrione, sulfosate,
tar oils, TCA, TCA-sodium, tebutam, tebuthiuron, terbacil, terbumeton, terbutylazine, terbutryn, thiazafluoron, thifensulfuron, thiobencarb, thiocarbazil, tralkoxydim, triallate, triasulfuron, tribenuron, triclopyr, tridiphane, trietazine, trifluralin, tycor, thi-diazimin, thiazopyr, triflusulfuron,
vernolate.

Preferred algicides are triazine compounds, such as, for example, terbutryn, cybutryn, propazine or terbuton, urea compounds such as, for example, diuron, benzthiazuron, methabenzthiazuron, tebuthiuron, and isoproturon, or uracils, such as, for example, terbacil.

In one variant of the invention the mixtures according to the invention do not contain either pyrithione or salts thereof.

The mixtures according to the invention contain in addition at least one aliphatic primary polyamine which comprises at least 5 carbon atoms and does not bear any secondary, tertiary or quaternary amino groups.

Preferred aliphatic primary polyamines which comprise at least 5 carbon atoms and do not bear any secondary, tertiary or quaternary amino groups are:

non-cyclic C₅-C₁₀ 1,w-diamines such as 1,5-pentanediamine, 1,5-diamino-2-methylpentane, 1,6-hexanediamine, 1,7-heptanediamine, 1,8-octanediamine and 1,9-nonanediamine;
cyclic C₆-C₁₀-diamines such as 1,3-bisaminomethylcyclohexane and 1,3-xylylenediamine,
non-cyclic polyetheramines such as polyetheramines of the formulae I and II

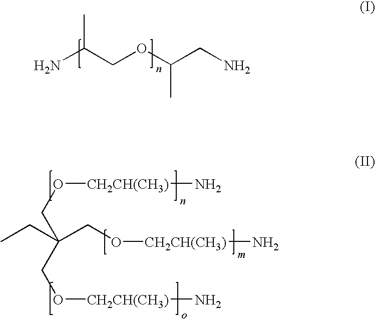

where
n or n, m and o in each case independently of one another is a natural number, preferably a natural number from 1 to 10, and particularly preferably 2 to 5,
   or mixtures of the abovementioned polyamines.

The abovementioned amines of the formulae I and II are, for example, commercially available as polyetheramine D230 and polyetheramine T403.

Particularly preferred aliphatic primary polyamines which comprise at least 5 carbon atoms and do not bear any secondary, tertiary or quaternary amino groups are: 1,5-diamino-2-methylpentane, 1,3-bisaminomethylcyclohexane and 1,3-xylylenediamine and also polyetheramines of the formulae I and II having the meanings mentioned there including their preferred ranges and also mixtures of the abovementioned polyamines.

The weight ratio of biocides to polyamines in the mixtures according to the invention can be, for example, 100:1 to 1:100, preferably 10:1 to 1:10, and particularly preferably 2:1 to 1:5.

Optionally, the mixtures according to the invention can additionally contain acidic substances. In the context of the invention, acidic substances are acids having a standard pK_{A} of 2.0 to 6.0 and salts thereof which, measured or calculated on an aqueous scale and a concentration of 1 mol/l, generate a pH of 2.0 to 6.0 at standard temperature.

Preferred acidic substances are: formic acid, acetic acid, propionic acid, butyric acid, succinic acid, lactic acid, citric acid, benzoic acid, adipic acid, fumaric acid, sorbic acid, salicylic acid, sorbic acid, nonanoic acid.

The mixtures according to the invention can additionally contain various additives. For the additives mentioned hereinafter, there is in each case, independently of each other, also the possibility that they are not present. Possible additives are, for example:
water. The mixtures according to the invention can contain, for example 0.01 to 98% by weight, preferably 5 to 90% by weight, of water. Suitable pHs under standard conditions range depending on the desired application from 2 to 11.
surface-active substances, for example surfactants. Surfactants can be, for example, nonionic, cationic and amphoteric surfactants, preferably anionic surfactants. Suitable anionic surfactants are, for example, alkyl sulfates, alkyl ether sulfates, alkyl aryl sulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefin sulfonates, in particular the alkali metal and alkaline earth metal salts, e.g. sodium, potassium, magnesium, calcium, and also ammonium salts and triethanolamine salts and ammonium salts of the abovementioned polyamines. The alkyl ether sulfates, alkyl ether phosphates and alkyl ether carboxylates can in each case have, for example, between 1 and 10 ethylene oxide or propylene oxide units, preferably 1 to 3 ethylene oxide units. Suitable substances are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecyl benzenesulfonate, triethanolamine dodecyl benzenesulfonate. The mixtures according to the invention can contain, for example 0.01 to 10% by weight, preferably 0.2 to 8% by weight, of surface-active substances.
emulsifiers such as, for example, sodium salts, potassium salts, ammonium salts and ammonium salts of the abovementioned polyamines of straight-chain aliphatic carboxylic acids of chain length C₁₂-C₂₀, sodium hydroxyoctadecanesulfonate, sodium salts, potassium salts and ammonium salts of the abovementioned polyamines of hydroxy fatty acids of chain length C₁₂-C₂₀ and sulfonation or acetylation products thereof, alkyl sulfates, also as triethanolamine salts, alkyl(C₁₀-C₂₀)sulfonates, alkyl(C₁₀-C₂₀)arylsulfonates, dimethyldialkyl(C₈-C₁₈)ammonium chloride, acyl-, alkyl-, oleyl- and alkylaryloxethylates and sulfonation products thereof, alkali metal salts of esters of sulfosuccinic acid with aliphatic saturated monohydric alcohols of chain length C₄-C₁₆, sulfosuccinic acid 4-esters with polyethylene glycol ethers of monohydric aliphatic alcohols of chain length C₁₀-C₁₂ (disodium salt), sulfosuccinic acid 4-esters with polyethylene glycol nonylphenyl ethers (disodium salt), sulfosuccinic acid bis-cyclohexyl ester (sodium salt), lignosulfonic acid and also calcium, magnesium, sodium and ammonium salts thereof, polyoxyethylene sorbitan monooleate having 20 ethylene oxide groups, resin acids, hydrogenated and dehydrogenated resin acids and also alkali metal salts thereof, dodecylated sodium diphenyl ether disulfonate and also copolymers of ethylene oxide and propylene oxide having a minimum content of 10% by weight of ethylene oxide. Preferably the emulsifiers used are: sodium lauryl sulfate, sodium lauryl ether sulfate, ethoxylated (3 ethylene oxide groups); the polyethylene glycol(4-20)ethers of oleyl alcohol and also the polyethene oxide (4-14)ethers of nonylphenol. The mixtures according to the invention can contain, for example 0.01 to 15% by weight, preferably 0.2 to 8% by weight, particularly preferably 0.5 to 6% by weight, and very particularly preferably 1 to 5% by weight, of emulsifiers.
dispersants, such as, for example, alkyl phenol polyglycol ethers. The agents according to the invention can contain, for example 0.01 to 8% by weight, preferably 0.1 to 6% by weight, particularly preferably 0.2 to 5% by weight, and very particularly preferably 0.2 to 3% by weight, of dispersants.
stabilizers, such as, e.g., cellulose and cellulose derivatives. The agents according to the invention can contain, for example 0.01 to 6% by weight, preferably 0.01 to 3% by weight, particularly preferably 0.01 to 2% by weight, and very particularly preferably 0.01 to 1% by weight, of stabilizers.
spreading agents, such as, for example, isopropyl myristate, polyoxyethylene nonyl phenyl ether and polyoxyethylene lauryl phenyl ether. The mixtures according to the invention can contain, for example 0.01 to 20% by weight, preferably 0.1 to 10% by weight, particularly preferably 0.1 to 5% by weight, and very particularly preferably 0.1 to 2% by weight, of spreading agents.
organic solvents such as, for example, monohydric or polyhydric alcohols, polyethers, polyether alcohols, esters, polyesters, polyester alcohols, ketones and hydrocarbons. The mixtures according to the invention can contain, for example, 0.01 to 95% by weight of organic solvent.
fragrances and dyes such as inorganic pigments, e.g. iron oxide, titanium oxide, ferrocyanide blue and organic dyes.
buffer substances, buffer systems or pH regulators. The mixtures according to the invention can contain, for example in each case 0.01 to 10% by weight, preferably 0.1 to 5% by weight, of buffer substances, buffer systems or pH regulators, the above-mentioned polyamines can in this case likewise act as a component of buffer systems which makes them valuable multifunctional components in the mixtures according to the invention.

The invention also comprises the mixtures according to the invention in any desired formulation. Preferred formulations are capsule suspensions (CS), water-soluble concentrates (SL), suspension concentrates (SC) and emulsifiable concentrates (EC), wherein water-soluble concentrates (SL), suspension concentrates (SC) and emulsifiable concentrates (EC) are generally preferred owing to the physical properties of the polyamines. In principle, suitable types of formulation essentially depend on the mixture components used and physical properties thereof. However, since these are known, it is familiar practice to those skilled in the art to determine further preferred manners of formulation in only a few experiments.

The particular advantage of the mixtures according to the invention is that the content of polyamines can improve the activity of the biocide component or components used and not deactivate, or deactivate only insignificantly, anionic surfactants which are frequently a component of biocidal formulations.

The invention therefore further relates to the use of the mixtures according to the invention for protection of industrial materials. Industrial materials which come into consideration are, in particular, adhesives, concrete, polymer dispersions, pigment slurries, inks, sizes, paints, coatings, plasters, cooling lubricants and heat transfer liquids and/or functional liquids for protecting the abovementioned industrial materials. Very particular preference is given to cooling lubricants and adhesives.

The invention in addition relates to a method for the protection of industrial materials against infestation and/or destruction by microorganisms, which is characterized in that the mixtures according to the invention are allowed to act undiluted or diluted on the microorganism or habitat thereof.

The invention in addition relates to industrial materials which are obtainable by treatment of industrial materials by the mixtures according to the invention.

Microorganisms which can effect degradation or change of the industrial materials which may be mentioned by way of example are bacteria, molds, yeasts, algae and slime organisms.

Preferably, the mixtures according to the invention are used against bacteria, molds and yeasts.

Microorganisms of the following genera may be mentioned by way of example:

Alternaria, such as Alternaria tenuis, Aspergillus, such as Aspergillus niger, Candids, such as candida albicans, Geotrichum such as Geotrichum candidum, Rhodotorula such as Rhodotorula rubra, saccharomyes such as Saccharomyces cerevisiae, Chaetomium, such as Chaetomium globosum, Coniophora, such as Coniophora puetana, Fusarium such as Fusarium solani, Lentinus, such as Lentinus tigrinus, Paecilomyces such as Paecilomyces variotti, Penicillium, such as Penicillium glaucum, Polyporus, such as Polyporus versicolor, Aureobasidium, such as Aureobasidium pullulans, Sclerophoma, such as Sclerophoma pityophila, Trichoderma, such as Trichoderma viride, Alcaligenes, such as Alcaligenes faecalis, Bacillus such as Bacillus subtilis, Escherichia, such as Escherichia coli, Proteus such as Proteus vulgaris, Pseudomonas, such as Pseudomonas aeruginosa and Pseudomonas fluorescens, Staphylococcus, such as Staphylococcus aureus.

The application rate of the mixtures according to the invention depends on the type and occurrence of the microorganisms to be controlled and also on the composition of the material to be protected. The optimum usage rate can be determined by test series simply and in a manner sufficiently known to those skilled in the art.

A further aspect of the invention relates to biocidal mixtures containing
at least pyrithione or a salt thereof
optionally at least one further biocide which is not an aliphatic primary polyamine and
at least one polyamine

Polyamines are all compounds which contain at least two amino groups. Preferred polyamines are those of the formulae III and IV

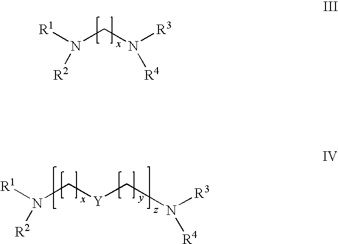

where
R¹, R², R³ and R⁴ can be hydrogen, saturated or unsaturated branched or linear C₁-C₁₀-alkyl, cycloalkyl, ethoxylates, propoxylates, alkylaryl or heterocyclyl
Y can be O, NH, NR¹, R¹—C═C—R², cycloalkyl, ethoxyl, propoxyl or CH₂
x, y and z are a natural number N

Examples of polyamines of the formulae III and IV are diethylenetriamine (CAS 111-40-0), bis(3-aminopropyl)ether (CAS 2579-20-6), 1,3-bis(aminomethyl)cyclohexane (CAS 2579-20-6), polyether amine (CAS 39423-51-3), diethylaminoethylamine (CAS 100-36-7), diethylaminopropylamine (CAS 104-78-9), N-aminopropylpiperidine and N-aminopropylmorpholine (CAS 123-00-2.

Further preferred polyamines are aliphatic primary polyamines which comprise at least 5 carbon atoms and do not bear any secondary, tertiary or quaternary amino groups, wherein the same ranges and preferred ranges apply as those which have been mentioned above.

In general, the above described disclosure, including the ranges and preferred ranges mentioned there, applies fully analogously to the pyrithione-containing mixtures with respect to further components such as further biocides, water, surface-active substances, emulsifiers, dispersants, stabilizers, spreading agents, organic solvents, fragrances and dyes, and also buffer substances, buffer systems or pH regulators.

As further biocides, however, in addition hexahydrotriazine (HHT), methylenebisoxazolidine (MBO), methylenebismorpholine (MBM), DMDM hydantoin (DMDMHy), tetrahydro-1,3,4,6-tetrakis(hydroxymethyl)imidazo[4,5-d]imidazole-2,5(1H,3H)-dione, benzisothiazolinone (BIT), o-phenylphenol (OPP), iodopropinylbutylcarbamate (IPBC) and mixtures thereof are particularly preferred.

It was also found, completely surprisingly to experts, that the polyamines spontaneously decolorize not only discolorations, e.g. in oil concentrates, but also in metalworking fluids, in the presence of heavy metals, in particular Fe-III ions, and also counteract in the long term a further discoloration. Such oil concentrates are generally made up as follows and can be mixed at room temperature in the stated sequence:

| up to 70% | base oil (naphthal-based, paraffin-based, |
|---|---|
| | e.g. Nynäs T22) |
| 20%-40% | emulsifier system/corrosion protection |
| | (petroleum sulfonates, potassium or |
| | amine soaps, nonionic surfactants, C₁₂-C₁₈ |
| | ethoxylates). Further emulsifiers are |
| | mentioned above. |
| up to 40% | solubilizer/water |
| 0.01-5.0%, | sodium pyrithione |
| preferably 0.1%-3% | |
| 0.01%-20% | polyamines and derivatives thereof |

This general formula of the present invention demonstrates the advantageous combination of pyrithione or salts thereof with polyamines. The necessary dosage of the polyamines is, for example, 0.2 to 10 times the concentration of sodium pyrithione and is, in particular, approximately the same concentration. The polyamines described according to the invention are suitable not only for preventing discolorations, but can also replace amines which must be used for the amine soaps given in the formula. The polyamines used can likewise contribute to the emulsification and to corrosion protection. The dosage of the polyamines can therefore advantageously be used in the emulsifier system/corrosion protection. The use of the polyamines described according to the invention is not restricted only to highly oil-containing cooling lubricants, but is universally suitable from semisynthetic products to clearly water-soluble complete syntheses.

The invention therefore comprises the use of polyamines for decolorizing and/or for preventing discolorations caused by heavy metal ions, in particular iron-III ions of substances containing pyrithione and/or salts thereof, preferably of oil concentrates, in particular cooling lubricant concentrates, paints and varnishes, adhesives, sealing compositions, leather auxiliaries, paper coating agents and cosmetics, and also the substances characterized by a fraction of one or more polyamine(s) and/or derivatives thereof.

In addition the invention comprises a method for decolorizing and/or preventing of discolorations caused by heavy metal ions, in particular iron-III ions, of substances containing pyrithione and/or salts thereof, by the addition or the provision of a fraction of one or more polyamines and/or derivatives thereof.

### EXAMPLES

### I Microbiological Examples

### Preservation of a Cooling Lubricant Emulsion

The preservative activity of the mixtures according to the invention was studied in a cooling lubricant emulsion (5% cooling lubricant concentrate on a mineral oil base/95% water). For this purpose, preserved samples of the cooling lubricant emulsion were repeatedly exposed in a weekly cycle to microbiological contamination with the following microorganisms:

| | |
|---|---|
| Bacteria: | Pseudomonas aeruginosa |
| | Pseudomonas fluorescens |
| | Pseudomonas oleovorans |
| | Pseudomonas rubescens |
| | Pseudomonas stutzeri |
| | Alcaligenes faecalis |
| | Citrobacter freundii |
| | Corynebacterium sp |
| Yeasts: | Rhodotorula rubra |
| Molds: | Acremonium strictum |
| | Fusarium solani |
| | Geotrichum candidum |

The bacteria mixture was added in each case separately from the yeasts/molds. The maximum experimental time was 10 weeks (=10 contaminations/inoculation cycles), provided that the weekly cell count determination (in each case 7 days after the contamination) was kept within the following limiting values:

bacteria <10⁶ CFU/g
yeasts/molds <10³⁻⁴ CFU/g

The experiment was terminated when said values were exceeded twice in succession.

### Example 1a

### For Comparison

Experiment using benzisothiazolinone as single component in a concentration of 0.05% benzisothiazolinone (=500 ppm or 2500 ppm of a commercially conventional 20% strength preparation) based on the mass of cooling lubricant emulsion.

Number of weeks in which the growth of microorganisms lay beneath the limiting values:

| | |
|---|---|
| Bacteria = | 1 week (of a maximum of 10 weeks) |
| Molds = | 1 week (of a maximum of 10 weeks) |
| Yeasts = | 7 weeks (of a maximum of 10 weeks) |

### Example 1b

### According to the Invention

Experiment with mixture according to the invention containing 20% by weight of benzisothiazolinone, 35% by weight of polyetheramine D230, an amine of the formula I having a molecular weight of approximately 230, 15% by weight of 1,5-diamino-2-methylpentane and 30% by weight of water. The abovementioned mixture according to the invention was used in an amount such that an amount of active substance of 0.05% (=500 ppm) of BIT in the cooling lubricant emulsion resulted:

Number of weeks in which the growth of microorganisms remained below the limiting values:

| | BIT/Amine | BIT |
|---|---|---|
| Bacteria = | 7 weeks | 1 week |
| Molds = | 1 week | 1 week |
| Yeasts = | 10 weeks | 7 weeks |

### Result:

The mixture according to the invention shows, compared with the single active ingredient BIT, significantly improved total activity, which is documented, in particular, by the significantly improved bacterial action.

### Example 2a

### For Comparison

Experiment using o-phenylphenol (OPP) as single component having an OPP concentration of 0.18% of OPP (=1800 ppm) based on the mass of cooling lubricant emulsion

Number of weeks in which the growth of microorganisms remained below the limiting values:

| | |
|---|---|
| Bacteria = | 2 weeks (of a maximum of 10 weeks) |
| Molds = | 10 weeks (of a maximum of 10 weeks) |
| Yeasts = | 10 weeks (of a maximum of 10 weeks) |

### Example 2b

### According to the Invention

Experiment using mixture according to the invention containing 55% by weight of OPP (45% by weight in water), 30% by weight of polyetheramine D230 and 15% by weight of 1,5-diamino-2-methylpentane.

The abovementioned mixture according to the invention was used in an amount such that an amount of active ingredient of 0.1% (=1000 ppm) of OPP in the cooling lubricant emulsion resulted:

Number of weeks in which the growth of microorganisms remained below the limiting values:

| | OPP/Amine | OPP |
|---|---|---|
| Bacteria = | 9 weeks | 2 weeks |
| Molds = | 10 weeks | 10 weeks |
| Yeasts = | 10 weeks | 10 weeks |

### Result:

The mixture according to the invention displays, compared with the single active ingredient OPP, significantly improved overall activity, which is documented, in particular, by the significantly improved bacterial action.

### II Examples on Decolorization

### Example 3

A fully synthetic cooling lubricant (clear, colorless liquid) or its 5% strength aqueous solution contains 0.1% of a preservative containing 10% sodium pyrithione. In the clear solution there are therefore 100 ppm of sodium pyrithione. 0.01 g of a 5% strength NH₄ Fe(III) (SO₄)₂ solution was added dropwise to this solution. An intensely deep-blue liquid was formed spontaneously. After addition of 0.05 g of diethylaminoethylamine, the solution was spontaneously decolorized.

Particularly suitable for the same purpose are:

diethylaminoethylamine CAS 100-36-7, diethylaminopropylamine CAS 104-78-9, N-aminopropylpiperidine and N-aminopropylmorpholine CAS 123-00-2.

### Example 4

In a clear solution of a cooling lubricant were present 100 ppm of sodium pyrithione. To this mixture were added dropwise 0.01 g of a 5% strength NH₄ Fe(III) (SO₄)₂ solution. A deep blue solution resulted immediately. After addition of 0.05 g of diethylenetriamine, immediately a water-clear liquid was obtained.

Particularly suitable for the same purpose are:

diethylenetriamine CAS 111-40-0, bis(3-aminopropyl)ether CAS 2579-20-6, 1,3-bis(aminomethyl)cyclohexane CAS 2579-20-6, polyetheramine CAS 39423-51-3

What is claimed is:

1. A biocidal mixture containing at least one biocide that is not an aliphatic primary polyamine and at least one aliphatic primary polyamine which has at least 5 carbon atoms and does not bear secondary, tertiary or quaternary amino groups, with those biocidal mixtures being excepted which contain diaminohexane and 1,2-benzisothiazolin-3-one, as are described in GB 1 330 531.

2. The biocidal mixture as claimed in claim 1, characterized in that the biocides that do not contain aliphatic primary polyamines are fungicides, bactericides and algicides.

3. The biocidal mixture as claimed in claim 1 or 2, characterized in that the biocides that do not contain aliphatic primary polyamines are those from the group consisting of azaconazole, bromuconazole, cyproconazole, dichlobutrazole, diniconazole, hexaconazole, metaconazole, penconazole, propiconazole, tebuconazole, dichlofluanid, tolylfluanid, triadimefon, fluorfolpet, methfuroxam, N-methylisothiazolin-3-one, 5-chloro-N-methyl-isothiazolin-3-one, dichloro-N-octylisothiazolinone, mercaptobenzothiazole, thiocyanatomethylthiobenzothiazole, thiabendazole, benzisothiazolinone, pyrithione and salts thereof, 2-bromo-2-nitro-1,3-propanediol and o-phenylphenol.

4. The biocidal mixture as claimed in any one of claims 1 to 3, characterized in that it does not contain either pyrithione or salts thereof.

5. The biocidal mixture as claimed in any one of claims 1 to 4, characterized in that the aliphatic primary polyamines which comprise at least 5 carbon atoms and do not bear any secondary, tertiary or quaternary amino groups are those which are selected from the group consisting of 1,5-pentanediamine, 1,5-diamino-2-methylpentane, 1,7-heptanediamine, 1,8-octanediamine and 1,9-nonanediamine, 1,3-bisaminomethylcyclohexane, 1,3-xylylenediamine and noncyclic polyether amines of the formulae I and II 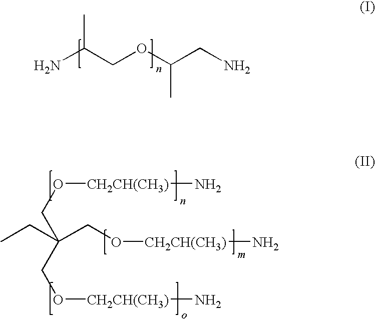
where
n or n, m and o in each case independently of one another is a natural number,
and also mixtures of the above polyamines.

6. The biocidal mixture as claimed in any one of claims 1 to 5, characterized in that the weight ratio of biocides to polyamines is 100:1 to 1:100.

7. The biocidal mixture as claimed in any one of claims 1 to 6, characterized in that it additionally contains acidic substances.

8. The biocidal mixture as claimed in any one of claims 1 to 7, characterized in that it is formulated as capsule suspension (CS), water-soluble concentrate (SL), suspension concentrate (SC) and emulsifiable concentrate (EC).

9. The use of biocidal mixtures as claimed in any one of claims 1 to 8 for protecting industrial materials.

10. The use as claimed in claim 9, characterized in that industrial materials are adhesives, concrete, polymer dispersions, pigment slurries, inks, sizes, paints, coatings, plasters, cooling lubricants and heat transfer liquids and/or functional liquids for protecting the abovementioned industrial materials.

11. A method for the protection of industrial materials against infestation and/or destruction by microorganisms, characterized in that the biocidal mixtures as claimed in any one of claims 1 to 8 are allowed to act undiluted or diluted on the microorganism or habitat thereof.

12. The method as claimed in claim 11, characterized in that microorganisms are bacteria, fungi, yeasts, algae and slime organisms.

13. The industrial materials obtainable by treating industrial materials with biocidal mixtures as claimed in any one of claims 1 to 8.

14. A biocidal mixture containing at least pyrithione or a salt thereof and at least one polyamine.

15. The biocidal mixture as claimed in claim 14, characterized in that the polyamines are selected from those of the formulae III and IV 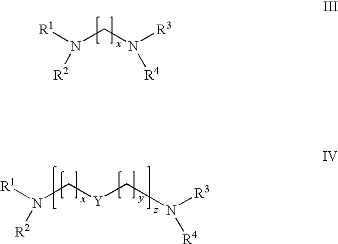
where
R¹, R², R³ and R⁴ can be hydrogen, saturated or unsaturated branched or linear C₁-C₁₀-alkyl, cycloalkyl, ethoxylates, propoxylates, alkylaryl or heterocyclyl
Y can be O, NH, NR¹, R¹—C═C—R², cycloalkyl, ethoxyl, propoxyl or CH₂
x, y and z are a natural number N.

16. The biocidal mixture as claimed in claim 14 or 15, characterized in that the concentration of the polyamine or polyamines and/or derivatives thereof is 0.2 to 10 times the concentration of pyrithione and/or the salt thereof.

17. The biocidal mixture as claimed in any one of claims 14 to 16, characterized in that, as further biocides, at least one is present which is selected from the group hexahydrotriazine (HHT), methylenebisoxazolidine (MBO), methylenebismorpholine (MBM), DMDM hydantoin (DMDMHy), tetrahydro-1,3,4,6-tetrakis(hydroxymethyl)imidazo[4,5-d]imidazole-2,5(1H,3H)-dione, benzisothiazolinone (BIT), o-phenylphenol (OPP), iodopropinylbutylcarbamates (IPBC) and mixtures thereof.

18. The use of polyamines and/or derivatives thereof for decolorizing and/or preventing discolorations caused by heavy metal ions of pyrithione and/or substances containing salts thereof.

19. The use as claimed in claim 18, characterized in that the concentration of the polyamine or polyamines and/or derivatives thereof is 0.2 to 10 times the concentration of the pyrithione and/or the salt thereof.

20. The use as claimed in claims 18 and 19, characterized in that the substances are oil concentrates, in particular cooling lubricant concentrates, paints and varnishes, adhesives, sealing compositions, leather auxiliaries, paper coating agents and cosmetics.

21. The use as claimed in any one of claims 18 to 20, characterized in that the substances are cooling lubricant concentrates and contain a base oil fraction up to 70% by weight, an emulsifier from 20 to 40% by weight, a solubilizer up to 40% by weight, and also 0.01 to 5.0% by weight of sodium pyrithione and polyamines having a fraction of 0.01 to 20% by weight, in each case based on the total amount.

22. A method for decolorizing and/or preventing discolorations caused by heavy metal ions of pyrithione and/or substances containing salts thereof characterized by the addition or provision of a fraction of one or more polyamines and/or derivatives thereof.

23. A substance containing a biocidal mixture as claimed in any one of claims 15 to 17.
